(19) United States
(12) Patent Application Publication
(10) Pub. No.: US 2009/0113984 A1
(43) Pub. Date: May 7, 2009

(54) GAS SENSOR SYSTEM HAVING A ZEROING MECHANISM

(75) Inventors: Steven Gautieri, Gladstone, MO (US); Peter Tobias, Minnetonka, MN (US)

Correspondence Address: HONEYWELL INTERNATIONAL INC., 101 COLUMBIA ROAD, P O BOX 2245, MORRISTOWN, NJ, 07962-2245, US

(73) Assignee: HONEYWELL INTERNATIONAL INC., Morristown, NJ (US)

(21) Appl. No.: 11/936,546

(22) Filed: Nov. 7, 2007

(51) Int. Cl. G01N 33/00; B01J 19/00

(52) U.S. Cl. 73/1.07; 422/83; 422/68.1

(57) ABSTRACT

A sensing system having a zeroing mechanism incorporating a micropump. The micropump may be connected to a gas conditioner for providing zeroing gas to a sensor downstream. The micropump and sensor may be connected to a processor or computer for pump control and receipt of information from the sensor. Upon zeroing and/or baseline correction of the sensor, the pump may be stopped and a sample gas may be fed into the sensor for detection and analysis. Alternatively, the pump may situated downstream of the sensor to draw conditioned gas through the sensor. A valve may be added having a conditioned gas input connected to the conditioner and having a sample input. The valve may have an output connected to the sensor. When the valve is open to the conditioned gas input, it will be closed to the sample input, and vice versa. The system may be for zeroing and/or calibration.

### BACKGROUND

The invention pertains to gas sensors, and particularly to zeroing gas sensors.

### SUMMARY

The invention is a gas sensor having a zeroing mechanism using a micropump and conditioner.

### BRIEF DESCRIPTION OF THE DRAWING

FIGS. 1a and 1b are diagrams of a system for a gas sensor having a zeroing mechanism having a micropump;

FIGS. 2a and 2b are diagrams of a system like that in FIGS. 1a and 1b having a location of the pump needing a valve;

FIG. 3 is a diagram of a conditioner using a fluid for conditioning gas that may be used in zeroing the sensor;

FIG. 4 is a diagram of a conditioner using a material such as carbon or another material for conditioning the gas;

FIG. 5 is a schematic illustration of an electrostatically actuated mesopump; and

FIG. 6 is an enlarged schematic view of one cell of the mesopump shown in FIG. 5.

### DESCRIPTION

Potentiometric gas sensors without reference gas, like functionalized FETs (FFETs), suffer from zero-gas signal drift (baseline drift). A baseline is a signal from a sensor when no analyte present, but otherwise under standard conditions. In most types of sensors, one significant limitation on a sensor's sensitivity is a gradual drift in its baseline which is a sensor's output when no sample gas is present. One way to improve the effective sensitivity of a sensor is to periodically measure its baseline output. This baseline may be subtracted from routine measurements to yield a signal which more faithfully represents the sample. However, if the sensor is to be used away from the laboratory, there may only a few practical ways measure the baseline. One way is to use a conditioner or filter. Such conditioner or filter may provide a stream of clean or conditioned air or gas which is passed to the sensor. Gas may refer to air and/or gas. Conditioned gas may refer to and include filtered, cleaned, humidified, analyte-added or -subtracted, and/or other property of a reference gas appropriate for zeroing, calibration, and baseline establishment of a sensor.

To evaluate the sensor signals, one should establish and occasionally reestablish a zero-gas signal. Zero as an adjective may refer to a condition where the sensor is exposed to neither analyte nor interference. The analyte may be of the gas that one is setting out to measure. A sample may be the gas being tested or analyzed for the presence of the analyte. The gas sample may contain an interference. An interference may include other gases that interfere with or affect the accuracy of a measurement of the analyte gas. The term “fluid” may refer to a liquid or gas.

Zero may be a relative term; for example, zero gas may refer to gas that is clean enough in that it does result in or affect a response under the conditions of a measurement. There may be a zero filter or conditioner that removes analyte and interfering gases from ambient air or gas. The conditioner may provide the conditioned air or gas used to set the baseline of a gas sensor.

By occasionally pumping gas through a filter or conditioner and then over the sensor, the ambient of the sensor may be replaced with conditioned gas. An example conditioner may include carbon, zeolite, or absorptive material for filtering and/or conditioning, a water rich material (as a filter for ammonia sensors), or other appropriate material. The conditioner may be preloaded with some water and possibly other vapors to avoid a gas over the sensor with zero humidity or zero other analyte, as applicable, which could lead to aberrant sensor signals.

On certain occasions, gas may be pumped over the sensor, coming from a pump and a conditioner, with either the pump or conditioner first. The flow may be rather small if the sensor is normally diffusion-based and the grids are properly designed and positioned. A potential issue with such approach is the cost of the pump. Regular pumps are too expensive and thus not practical for the present approach and system. An inexpensive micropump is desired. New technologies may make micropumps inexpensively possible. An example may be a Mesopump™ (being a trademark of and available from Honeywell, and referred to as a “mesopump” herein) which is a micropump of quite low cost, uses little power and provides a low flow rate. Such micropump appears suitable for the present task.

Gas from a pump may lead to a zero signal of the sensor, which can be obtained even without waiting for a settled signal if a predictive algorithm is used and the signal follows a known behavior like an exponential decay.

A significant factor of the present approach is that a micropump and filter may be combined with a single sensor. The single sensor could be an array of various sensors for detecting and/or measuring different parameters at one point, spot or location. The low cost of a micropump such as a mesopump may make this combination possible and practicable. An illustrative example of mesopump is shown in U.S. Pat. No. 6,106,245, issued Aug. 22, 2000, which is owned by the assignee of the present application, and hereby incorporated by reference. Certain aspects of the mesopump may be noted. The present system may incorporate a specific application of this pump.

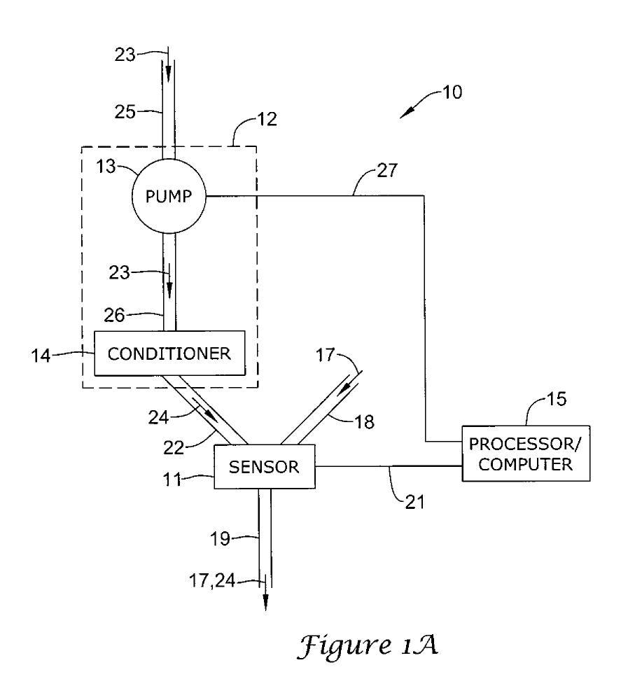

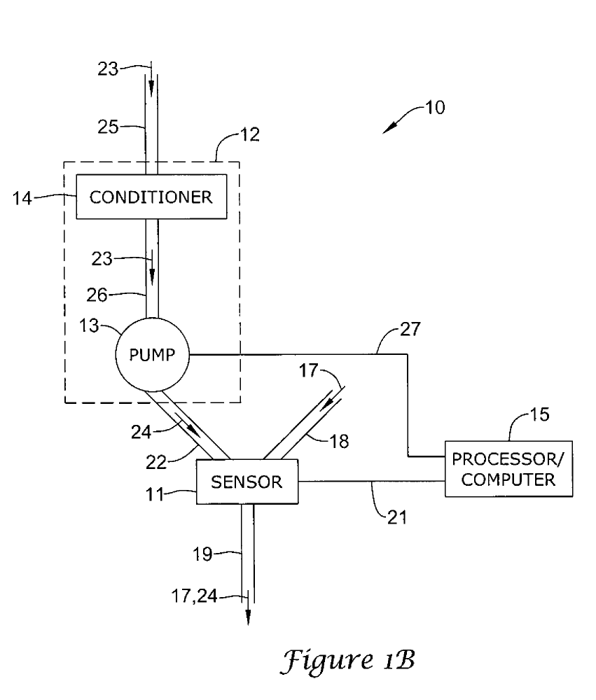

FIGS. 1a and 1b are diagrams of a system 10 for a gas sensor 11 having a zeroing mechanism 12. There may be other configurations of system 10 besides those shown in FIGS. 1a and 1b. The zeroing mechanism may include a pump 13, such as a micropump or mesopump, and a conditioner 14. System 10 may also include a processor or computer 15 connected to sensor 11 via line 21. The processor or computer 15 may also be connected to pump 13 via a line 27.

A sample 17 of analyte may enter an input 18 to sensor 11 for detection. The detected or analyzed sample may exit the sensor 11 via an output 19. Information about the gas may go to the processor 15 in a form of an electrical signal on a line 21. However, if no sample is run through the sensor 11, an output from sensor 11 may indicate something of a sample which is not present in the sensor. This signal output 21 may reflect a shift in a baseline. With no sample 17 in the sensor, the signal should reflect an absence of the sample if the baseline is appropriate. To check and set the baseline of sensor 11, the sensor 11 may be zeroed. For instance, a conditioned zeroing gas 24 may be provided through an input 22 to sensor 11. There may be various sources of gas 24, especially in a laboratory setting. The present system 10 may include a sensor 11 in a remote location, so providing a clear or conditioned zeroing gas in an inexpensive and convenient way is important.

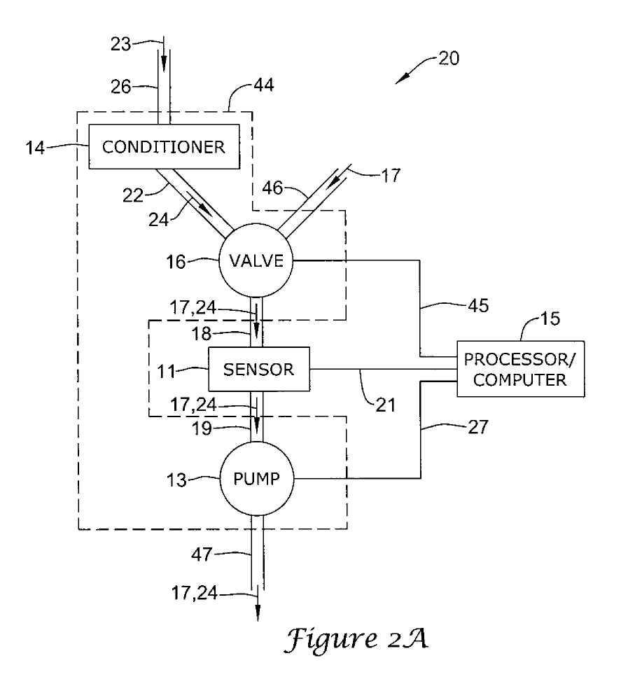

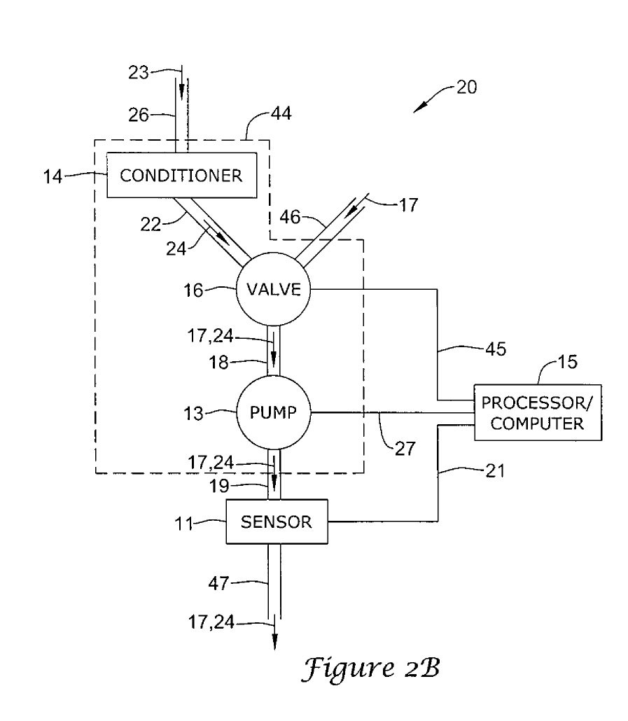

The conditioning or zeroing mechanism 12 may provide a gas 24 to sensor 11 for zeroing, calibration and appropriate baseline establishment. Pump 13 may pull in air or a portion of the sample 17 as a gas 23 to an input 25 of the pump. Pump 13 may be turned on as needed by a signal along connection 27 from processor 15 to move the gas 23 through an input 26 to the conditioner 14. Pump 13 as noted herein may be a micropump such as an inexpensive mesopump. It may be just a one unit or one cell mesopump. Since gas 23 may not be suitable for zeroing the sensor 11, gas 23 may be conditioned into a gas 24 suitable for zeroing. The order and location of the pump 13 and conditioner 14 may vary as shown by illustrative examples of system 10 in FIGS. 1a and 1b and system 20 of FIGS. 2a and 2b. Gas may be pushed or pulled over the conditioner and sensor. Pump 13 may be placed in virtually any place in the system generally where workable and practical. Pump 13 may be a low-power micropump used typically for calibration within short periods of time in many instances. Also, when the micropump 13 is used for moving a sample over a sensor, the power and time used may be rather minimal. For the present system, the shelf life of the micropump would tend to be significantly longer than the active lifetime of the micropump.

Conditioner 14 may be reconditioned by flowing a heated gas backwards through the conditioner. The gas flowed backwards through the conditioner is not to be flowed over or through the sensor. This approach for reconditioning is not necessarily applicable to conditioners of a liquid version, such as a water solution conditioner.

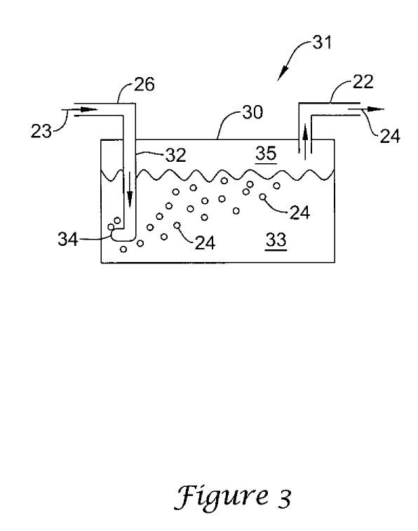

Conditioner 14 may be a filter. An instance of filtering may include gas 23 being pumped through another fluid for conditioning such as in an approach shown in FIG. 3. This Figure is a diagram of a filter 31 which may have a container 30 with input 26 and output 22 connected to the container. Input 26 may be connected to a tube 32 which is inserted in a fluid 33. A porous plug 34 may be placed at the lower end of tube 32. Gas 23 may be pushed through the porous plug 34 and cause the gas to bubble through the fluid 33 up into a space 35. The gas may become cleansed or conditioned gas 24 which may fill space 35 and be forced out of filter 31 via tube 22 to sensor 11 for zeroing, baseline correction, and/or calibration. The gas 24 may exit the output 19 of sensor 11. Processor 15 may take note of the latter when completed and then turn off pump 13 via line 27. Sensor 11 may resume normal activities of detection and/or analysis of gas 17 entering input 18 of sensor 11 and exiting output 19. For maintaining the full potential of sensor 11, its baseline may be measured periodically (e.g., every 30 minutes) and the sensor be zeroed as necessary.

Filter 31 may contain a fluid 33 such as a saturated salt solution, water with a table salt, or other solution. Humidity should be present in the fluid of container 30. Low humidity such as five percent may be sufficient. A saturated salt solution may provide a certain amount of humidity which is indicated by the kind of salt in the solution. Plain water as the fluid 33 may provide too much humidity to the gas 24. Such humidity in the conditioned gas 24 may approach 100 percent and result in condensing. However, an addition of salt to the water may reduce and set the humidity of water-conditioned gas and eliminate condensing issues. A good starting point for humidity may be about 50 percent. For an example, adding table salt to the water may result in about 60 percent in the conditioned gas. A moderate amount of humidity would be an amount sufficient for adequate operation and baseline setting of the sensor and not resulting in condensing issues.

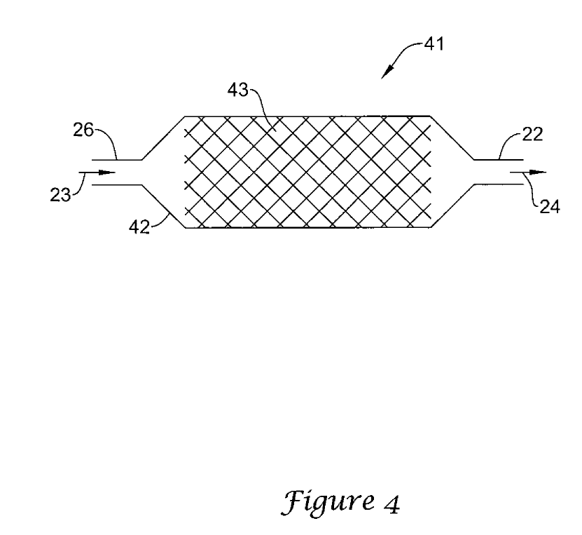

Conditioner 14 may contain a filter 41 having a container 42 filled with a carbon, zeolite, and/or other material 43, as shown in FIG. 4. Gas 23 may enter filter 41 through tube 26. Gas 23 may go through the carbon and/or material 43 and become conditioned gas 24 and be forced out of filter 41 via tube 22 to sensor 11 in the same manner relative to filter 31 with the subsequent action relative to sensor 11. Filter 41 may have material as needed to provide appropriate humidity to the filtered gas to be used for zeroing and baseline calibration of sensor 11.

FIGS. 2a and 2b are diagrams of a system 20 having rearrangements of components of system 10 plus a valve 16. There may be other configurations of system 20. The pump 13 may be situated downstream from a point of entry for a sample gas 17. System 20 may have a conditioning mechanism 44 which may include conditioner 14, valve 16 and pump 13. Pump 13 may be before or after sensor 11 according to the direction of flow. System 20 may have a processor or computer 15 connected to sensor 11 via line (or connection) 21. Also, processor/computer 15 may be connected to pump 13 via line 27 and to valve 16 via a line 45. A gas 23 may enter conditioner 14 at input tube 26 as it may be drawn by a downstream pump 13. Gas 23 may be conditioned to be a zeroing gas for sensor 11 and an adjustment or recalibration of its baseline. Filter 31, 41 or other type of filter may accomplish the preparation of gas 23 at conditioner 14 into a zeroing gas 24. Gas 24 may exit conditioner 14 in tube 22. Gas 24 may go through valve 16 that provides an open passage way from tube 22 to the input tube 18 and eventually to sensor 11. At about the same time, the passage from an input tube 46 to input tube 18 may be closed. Gas 24 may enter sensor 11 for zeroing, calibration and appropriate baseline establishment. Gas 24 may exit sensor 11 through tube 19 or 47, pulled or pushed respectively, by pump 13. Pump 13 may expel gas 24 from system 20 via an output tube 47. Upon completion of zeroing, calibration and baseline establishment by processor/computer 15 with gas 24, processor/computer 15 may provide a signal on line 45 to valve 16 to close the passage way between tube 22 from conditioner 14 to input tube 18. At about the same time, the signal on line 45 may open a passage way between input tube 46 to valve 16, and tube 18 from valve 16 to an input of sensor 11 or pump 13 for permitting a sample gas 17 to go through sensor 11 for detection and analysis. A signal or signals representing the detection and analysis information may go via line 21 to processor/computer 15 for processing, saving, plotting, analysis, and/or the like. The detected and analyzed gas 17 may be drawn or pushed out of sensor 11 via a tube 19 or 47, respectively, by pump 13. Pump 13 may expel gas 17 from system 20 via an output tube 47. Operation of pump 13 may be controlled by processor/computer 15 via line 27.

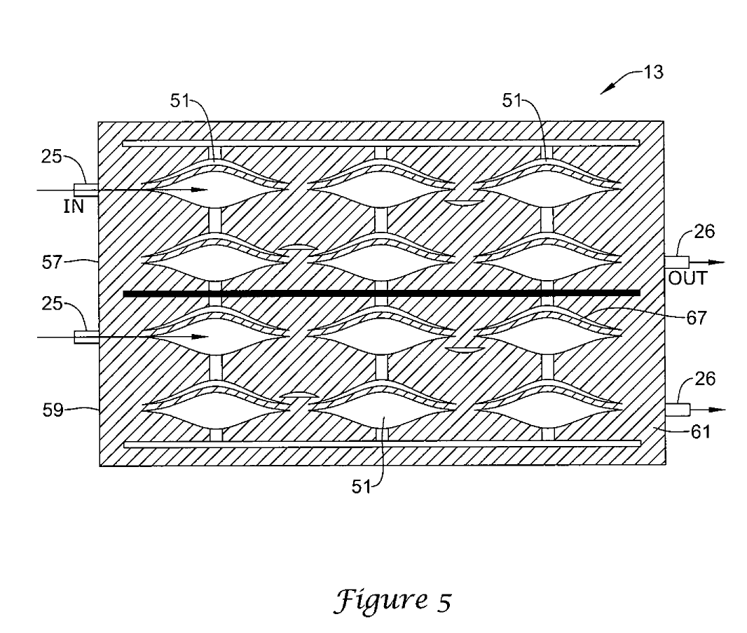

FIG. 5 illustrates an example micropump 13 that may be used in systems 10 and 20. This micropump 13 may be instantiated with a mesopump. The mesopump is just an example among other pumps that may be used in the present system. The mesopump may be realized as a single unit or as an array of up to 100 parallel units or channels, so that pumping rates may be achieved from less than 1 milliliter/min to about 10 liters/min. By using electrostatic actuation, the power consumption may be kept below 5 mV/channel and below 0.5 W per 100-channel array. The actuation voltages may be kept below 50 volts, particularly because of the specific shape of the electrodes. As an example, a 100 channel array may have a size of only one cubic inch. A single unit or channel may have proportionately a much smaller size.

The mesopump 13 could consist of a plurality of cells 51 that transfer fluid from an inlet 25 to an outlet 26. Mesopump 13 may have an upper channel 57 and a lower channel 59, arranged in a parallel relationship, with both channels functioning in the same manner.

The body 61 may be constructed by molding a high temperature plastic such as ULTEM™ (trademark of General Electric Company, Pittsfield, Mass.), CELAZOLE™ (trademark of Hoechst-Celanese Corporation, Summit, N.J.), or KETRON™ (trademark of Polymer Corporation, Reading, Pa.). The electrodes themselves may be formed by printing, plating or electron beam (EB) deposition of metal followed by patterning by using dry film resist. A low temperature organic and/or inorganic dielectric may be used as an insulator between the actuating electrodes.

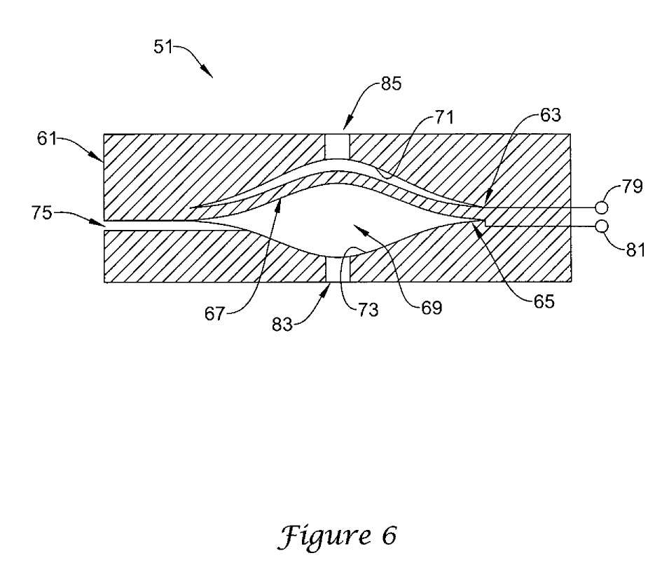

As may be shown in FIG. 6, each cell 51 of the mesopump of FIG. 5 may have a molded pump body 61 with an upper actuation electrode 63 and a lower actuation electrode 65. Body 61 may also mount an electrically grounded diaphragm 67 such that diaphragm 67 is capable of movement inside chamber 69 between upper electrode curved surface 71 and lower electrode curved surface 73. Body 61 may also include an inlet lateral conduit 75 which may be input 25 and an outlet conduit which could be an output 26.

Diaphragm 67 may conform to curved surfaces 71 and 73 when it is electrostatically driven to one or the other surfaces through application of a voltage to the particular electrode via voltage source 79 for upper electrode 63 and voltage source 81 for lower electrode 65. Diaphragm 67 and the curved surfaces 71 and 73 may be coated with thin dielectric layers at for electrical insulation and protection.

The mesopump body 61 may also include a vertical conduit 83 in curved surface 73 which permits material in chamber 69 between diaphragm 67 and the lower electrode 65 to be discharged when voltage is applied to move diaphragm into substantial contact with surface 73. Body 61 may also include a back pressure control conduit 85 in the upper electrode curved surface 71.

In the present specification, some of the matter may be of a hypothetical or prophetic nature although stated in another manner or tense.

Although the invention has been described with respect to at least one illustrative example, many variations and modifications will become apparent to those skilled in the art upon reading the present specification. It is therefore the intention that the appended claims be interpreted as broadly as possible in view of the prior art to include all such variations and modifications.

What is claimed is:

1. A sensing system comprising:
a gas sensor;
a conditioner for providing a conditioned gas; and
a micropump is for moving the conditioned gas through the conditioner; and
wherein the conditioned gas is for zeroing and/or calibration of the sensor.

2. The system of claim 1, wherein the micropump is a mesopump.

3. The system of claim 2, wherein the mesopump is a single cell pump.

4. The system of claim 1, wherein:
the micropump is for a single sensor; and
the single sensor has one or more sensors for detecting and/or measuring one or more parameters at one spot.

5. The system of claim 1, wherein zeroing the sensor provides a basis for setting a baseline of the sensor for during no detection of a sample of analyte.

6. The system of claim 5, further comprising a processor connected to the micropump and the sensor.

7. The system of claim 1, wherein the micropump is for moving a gas through the sensor.

8. The system of claim 1, wherein the sensor comprises an input for a sample gas.

9. The system of claim 8, wherein if the micropump is not moving a conditioned gas through the sensor, then the sensor may receive a sample gas for detection and/or analysis.

10. The system of claim 1, wherein the conditioner comprises a container having a fluid through which a gas for conditioning goes.

11. The system of claim 10, wherein the fluid is for bubbling gas through it to be conditioned.

12. The system of claim 10, wherein the container has an input which has a porous plug situated in the fluid for bubbling gas through the fluid.

13. The system of claim 10, wherein the fluid is a saturated salt solution for conditioning the gas and providing humidity to the gas in an adequate amount but not so much as to result in a condensing gas.

14. The system of claim 1, wherein the conditioner comprises a filter comprising at least one of a group containing carbon, zeolite or absorptive material.

15. The system of claim 14, wherein the conditioner is for adding a moderate amount of humidity to a gas being conditioned.

16. A sensor system comprising:
a valve having a zeroing fluid input, a sample fluid input, and an output;
a fluid sensor having an input ultimately connected to the output of the valve; and
a micropump for moving fluid through the conditioner and fluid sensor; and
wherein the output of the valve is switchable between the zeroing fluid input and the sample fluid input.

17. The sensor of claim 16, wherein a conditioned fluid can be drawn through the zeroing fluid input, valve and sensor to set a baseline for a zero sample fluid input.

18. The sensor of claim 16, wherein:
the fluid is a gas; and
the mesopump is a single unit pump.

19. A method for zeroing a gas sensor comprising:
providing a gas sensor;
pumping a conditioned gas through the sensor;
resetting a baseline of the sensor relative to a zero gas signal from the sensor; and
wherein the pumping is provided with a micropump.

20. The method of claim 19, wherein the micropump is a single cell mesopump.
